(19) United States
(12) Patent Application Publication
(10) Pub. No.: US 2006/0253190 A1
(43) Pub. Date: Nov. 9, 2006

(54) Removeable stents

(76) Inventor: Michael D. Kuo, San Diego, CA (US)

Correspondence Address: Dennis A. Bennett, Esq., 560 Legends View Drive, Eureka, MO, 63025, US

(21) Appl. No.: 11/429,679

(22) Filed: May 8, 2006

Related U.S. Application Data
(60) Provisional application No. 60/678,843, filed on May 6, 2005.
(60) Provisional application No. 60/693,332, filed on Jun. 22, 2005.

(51) Int. Cl. A61F 2/06

(52) U.S. Cl. 623/1.44

(57) ABSTRACT

There is disclosed novel removable stent grafts and methods for removing stent grafts. There is also disclosed new diseases and disorders that can now be treated with removable stents.

### RELATED APPLICATION(S)

This Application claims priority of U.S. provisional application Ser. No. 60/678,843, filed May 6, 2005 and U.S. provisional application Ser. No. 60/693,332, filed Jun. 22, 2005.

### FIELD OF THE INVENTION

This invention relates to the field of intraluminal grafts and particularly to thin-wall intraluminal grafts useful as an inner lining for blood vessels or other body conduits and methods to remove them.

### BACKGROUND OF THE INVENTION

The percutaneous management of biliary strictures remains poorly defined. Treatment has classically been guided by whether the underlying disease process is benign or malignant. In cases of inoperable malignant disease, bare metal stents are routinely used as the desire for biliary conduit restoration outweighs their relatively poor overall primary patency due to tissue in-growth. In contrast, the poor long term performance of metallic stents in the biliary system and their inability to be removed limits their widespread application in the treatment of benign biliary disease. Subsequently, percutaneous management has required repeated balloon dilation and long term biliary drain placement.

To date, covered stents have proven feasible in the treatment of malignant biliary disease. Applications in the biliary system were initially developed to address the issue of tissue in-growth. The covered stent provides a mechanical barrier that limits tissue in-growth and helps maintain intra-stent patency. But, complications such as infection, inadvertent “jailing” of bile ducts, intra-stent sludge accumulation or foreign body occlusion still represent very real problems.

The lack of tissue in-growth with stent grafts affords a possible additional advantage of potentially allowing for device retrieval. This provides a solution for problems that plague current stent utilization including tissue in-growth, infection, sludge accumulation, foreign body occlusion, device misplacement, and may also even obviate the need for long term drain requirement. A retrievable stent graft also affords the operator greater latitude in determining when and whom to stent, which may be of particular benefit in cases where the underlying lesion is not well characterized. In addition, it is possible that a retrievable stent graft could provide significant therapeutic benefits by providing a temporary scaffold around which the lesion can remodel. These same principles apply in general to all types of disease processes in which stents and or stent grafts can be applied.

The novel concept of removable stents for the treatment of biliary disease is largely based on the idea of providing a biliary conduit to treat or prevent stricture formation while simultaneously offering the flexibility of later removal when no longer needed or desired. Plastic stents have been routinely used in such settings but unfortunately, experience high rates of malfunction, occlusion and migration. Alternatively, certain groups have reported placing and later removing bare metal stents in a small number of cases. However, tissue in-growth, inucosal hypertrophy and stent incorporation are very serious and real problems for potential retrieval, particularly in patients with a long life expectancy and thus are generally not advocated for treatment of benign biliary disease. Conversely, covered stents have been employed in the biliary system to address the problems of tissue in-growth and have proven feasible in the treatment of inoperable malignant disease. However, stent grafts in the biliary system can still be complicated by infection or occlusion; thus, the need for removal in this population still exists. Fortunately, as the graft material, and in particular ePTFE, serves both as an effective barrier to tissue in-growth and as has been discovered with these studies is a relatively friction-free surface, this makes these devices particularly well-suited for potential removal.

Petersen et al initially described their experience with a home-made retrievable biliary stent graft in a small number of patients. Their device consisted of ePTFE covered Gianturco-Rosch Z stents (self expanding stainless steel stents) with a retrieval suture affixed to the proximal aspect of the stent graft. In their study, they were able to successfully retrieve 9 of 9 stent grafts from 7 patients at up to 9 months which suggests that tissue in-growth was not a rate limiting factor in device retrieval. However, they noted a 27% incidence (3/11) of device migration in their series. Further, they also reported several cases of device malfunction including rupture of the retrieval-suture in 2 out of 9 (˜22% device retrieval apparatus failure) of their devices which ultimately required tract upsizing and piecemeal extraction of the stent grafts.

While this study work demonstrates the potential feasibility of intentional percutaneous retrieval of stent grafts from the biliary system there are a number of limitations with this approach. Clearly, the tendency for device migration inherent in this device construction is a significant primary limitation; if the device can not stay in the original location where it is needed to exert its action and thus cannot even perform its primary function, than the need to retrieve it at a later time once it has migrated is clearly immaterial. Additionally, the use of a retrieval suture as the primary means of retrieving the stent graft is prone to failure as was seen in these cases where the suture ruptured in 22% of cases. The prominent thickness of the ePTFE used (8-10 mm diameter), and the construction of this stent graft using Gianturco-Rosch Z stents is also a limitation for retrieval in that the primary structure and design of the Gianturco-Rosch stents, the method in which they are appended together (individual Z stents held together in tandem by an outer coating of manually sutured ePTFE) as well as the composition of the stent material (stainless steel), all do not allow for optimal re-compression of the stent grafts once captured, as tract upsizing and piecemeal extraction of the stent grafts was not uncommon in their series. Further, the porosity of the ePTFE used in these devices does not limit or impede intra-stent sludge accumulation, which can represent a significant obstacle to retrieval as this may effectively increase the total stent graft retrieval volume, thereby making it even harder to re-compress it into the retrieval sheath or may even facilitate adherence of the stent graft to the native duct wall thereby increasing removal friction and the potential for significant iatrogenic injury. Additionally, the use of retrieval sutures that are exposed with in the native bile duct lumen increases the potential risk for proximal sludge or debris accumulation which may get caught in the retrieval sutures. This would then impede retrieval by denying access to the device due to the proximal obstruction created by this focus. Also, with their design, retrieval was performed in a number of cases by “pushing” the stent graft forward through the bile duct and into the bowel in order to gain access to, and capture the stent graft. This means of capturing, which again inherently relies on the device's tendency to migrate, can cause iatrogenic injury to the bile duct and surrounding structures as the stent graft is manually forced along the bile duct and into the bowel.

Similarly, endoscopic removal of covered Wallstents (polyester covered Ni—Co—Ti self expanding steel alloy stents) has also been reported in the literature by Kahaleh. In this study, the authors, similar to the work of Petersen, also noted that the covered Wallstent's propensity for migration, which while not ideal for biliary disease management may in fact be a beneficial property for subsequent retrieval. In their series they report successful endoscopic removal of 13/14 covered Wallstents in both benign and malignant biliary disease. Their primary removal technique was facilitated by snaring the protruding distal stent graft edge from the duodenum via an endoscope. Again, this study supports the concept that the ability of the graft component to resist tissue in-growth and to simultaneously serve as a relatively friction free surface are ideal qualities for a retrievable device. However, again, incidentally as with Petersen, the authors again note that the indication for removal of 3/14 (21%) stent grafts in their study was precisely for stent migration. Clearly, many of the inherent limitations of device retrieval described using Petersen's method above are recapitulated here again with the covered Wallstent. Further, the long term primary patency of covered wallstents compared to ePTFE covered stents and the Viabil Biliary Endoprosthesis in particular, is inferior due to the graft material composition which may make it more prone to sludge accumulation. Indeed, the covered Wallstent has been shown to incite greater neointimal hyperplasia and thrombus induction along the graft surface compared to uncovered wallstents and ePTFE covered stents, supporting the concept that the polyester graft material itself may not be an ideal material for treating stenoses or strictures due to its inherent nature to incite intra-stent graft tissue formation and deposition and induce thrombus formation, all of which as described above would significantly impair attempted stent graft retrieval. Further, the covered wallstent also has uncovered portions at the margins consisting of bare metal. While these modifications may in theory minimize device migration, they significantly deter retrieval due to tissue ingrowth into the uncovered components which can serve to anchor the device to the body.

Intraluminal vascular grafts were suggested as early as 1912 in an article by Alexis Carrel (Results of the permanent intubation of the thoracic aorta. Surg., Gyn and Ob. 1912; 15:245-248). U.S. Pat. No. 3,657,744 to Ersek describes a method of using one or more adjustable stents to secure a flexible fabric vascular graft intraluminally, the graft and stent having been introduced distally and delivered to the desired position with a separate delivery system.

Choudhury, U.S. Pat. No. 4,140,126, describes a similar method of repairing aortic aneurysms whereby a polyethylene terephthalate vascular graft is fitted at its ends with metal anchoring pins and pleated longitudinally to collapse the graft to a size small enough to allow for distal introduction.

Rhodes, U.S. Pat. No. 5,122,154 and Lee, U.S. Pat. No. 5,123,917, describe endovascular bypass grafts for intraluminal use which comprise a sleeve having at least two diametrically-expandable stents. Rhodes teaches that the sleeve material is to be made of conventional vascular graft materials such as GORE-TEX.®. Vascular Graft® (W. L. Gore & Associates, Inc., Flagstaff Ariz.) or lmpra.®. Graft (Impra, Inc. Tempe Ariz.). Both the GORE-TEX Vascular Graft® and Impra Graft® are extruded and longitudinally expanded PTFE tubes. Additionally, the GORE-TEX Vascular Graft® possesses an exterior helical wrapping of porous expanded PTFE film. The difficulty with the use of either the GORE-TEX Vascular Graft® or the Impra graft® as the sleeve component is that the relatively thick, bulky wall of the extruded, longitudinally expanded PTFE tubes limits the ability of the tube to be contracted into a small cross-sectional area for insertion into a blood vessel. For example, the wall thickness of a 6 mm inside diameter Thin Walled GORE-TEX Vascular Graft is typically 0.4 mm. The thinness of the wall is limited by the difficulty of manufacturing an extruded, longitudinally expanded tube having a thin wall of uniform thickness.

U.S. application 20020082675 discloses a tubular intraluminal graft in the form of a tubular diametrically adjustable stent having a tubular covering of porous expanded polytetrafluoroethylene.

There is disclosed in U.S. Pat. No. 6,881,220 (a.k.a. a Fluency tracheobroncial stent graft® by Bard) an endoluminal coil stent comprising a hollow tube, which allows the passage of fluid, formed into a series of loops or other known stent shapes. The stent is made of a shape memory metal such as nitinol. Shape memory metals are a group of metallic compositions that have the ability to return to a defined shape or size when subjected to certain thermal or stress conditions. Shape memory metals are generally capable of being deformed at a relatively low temperature and, upon exposure to a relatively higher temperature, return to the defined shape or size they held prior to the deformation. This enables the stent to be inserted into the body in a deformed, smaller state so that it assumes its “remembered” larger shape once it is exposed to a higher temperature (i.e. body temperature or heated fluid) in vivo. The stent can also be removed by flowing lower temperature fluids through the stent causing it to shrink thereby allowing it to be removed. This stent is complicated and numerous things could go wrong like leaking of the fluid or a kink being in the stent that would prevent the stent to shrink properly.

The ability to easily and safely remove the above mentioned stents have limited their widespread application to the treatment and management of a large number of diseases. The present invention provides a description of applications for removable stents and methods to safely remove stents from within the body once they have already been deployed.

### SUMMARY OF THE INVENTION

There is disclosed intraluminal stent grafts which are capable of being removed. There is also disclosed methods to remove the stent graft . The ability to remove stent grafts allows numerous other diseases or conditions to be treated with stents that have to date not been possible.

### BRIEF DESCRIPTION OF THE DRAWINGS

FIG. 1 is a schematic drawing of the Viabil Biliary Endoprosthesis;

FIG. 2 is a side view of two different constructions of the Viabil Biliary Endoprosthesis. Completely covered stent graft version (top), and fenestrated version (bottom);

FIG. 3 is a side view of unzipping of the biliary stent graft as an ePTFE/FEP-covered nitinol wire;

FIG. 4 is a side view demonstrating stent graft removal devices consisting of a sheath (top), grasping forceps (middle) and the stent graft (bottom);

FIG. 5 is a side view demonstrating the grasping forceps grasping the edge of the stent graft;

### DETAILED DESCRIPTION

Various modifications to the above mentioned VIABIL Biliary Endoprosthesis, Fluency Tracheobroncial Stent and other stent graft devices can be made to assist in allowing them to be more easily retrievable/removeable from the body post deployment.

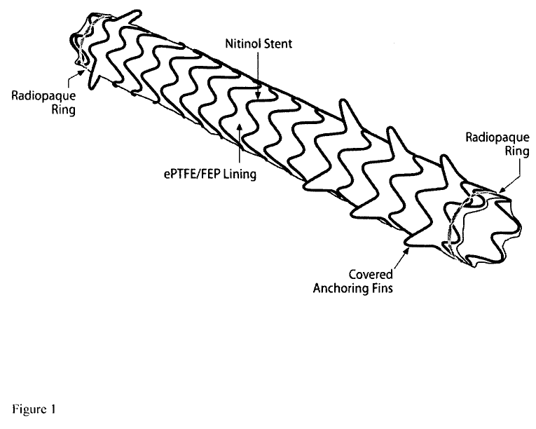

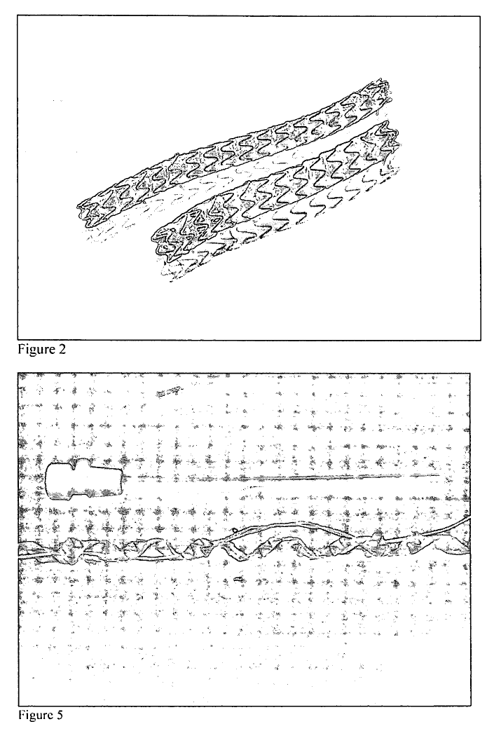

The Viabil Biliary Endoprosthesis stent graft described here is a dedicated biliary stent graft that employs an ultra thin non-porous ePTFE graft component. The design of the VIABIL Biliary Endoprosthesis® consists of a fluorinated ethylene propylene (FEP) powder coated, self expanding nitinol stent exoskeleton which is lined on it's inner surface with a tube composite of FEP impregnated expanded polytetrafluoroethylene (ePTFE) (FIGS. 1-2). This combination has demonstrated, in animal histology and human cadaver retrievals, that tissue does not in-grow to the external surface of the endoprosthesis. Subsequently, to prevent device migration, flexible nitinol wire loop anchoring fins are incorporated to maintain long-term device position after deployment. Likewise, the fins are covered with the same FEP/ePTFE composite material which prevents tissue encapsulation of the wire loop. The present invention takes advantage of this to minimize ductile tissue trauma in the event the physician deems removal necessary. This particular graft composition has increased resistance to bacterial colonization which is believed to be a contributing factor to intra-stent sludge accumulation, and has also been shown in ex-plant evaluations to resist tissue in-growth beyond 250 days. Further, it is demonstrated herein significantly improved primary patency rates compared to polyurethane-based stent grafts in the management of malignant biliary obstruction. To address the issue of stent migration this particular device employs bidirectional anchoring fins along both ends of the stent graft. In two studies evaluating a total of 68 patients treated for malignant biliary obstruction using this device, there were no reported observations of device migration at up to one year.

Several observations about this device, devices incorporating similar features or methods developed to remove this device suggest that they would ultimately be retrievable. First, given that the stent graft is completely covered, adherence and tissue in-growth should not represent a significant obstacle to attempted retrieval. Second, because the anchoring fins are relatively flexible and invertible they should not significantly impede retrieval of the device via either an anterograde or retrograde approach, whether by pushing or pulling the stent graft. Third, because of the relatively compliant and flexible, self expanding nature of the stent and its ultra thin graft fabric, the device is sufficiently compliant and should suitably deform to allow for relatively atraumatic extraction. Indeed, compared to other stent grafts, the Viabil is able to recompress to a small total volume with respect to its initial post deployment total volume. This in theory should facilitate its retrieval through a relatively narrow channel without requiring tract upsizing. Finally, it is noted that because the prosthesis in essence, consists of a single helically wound nitinol-ePTFE/FEP coated wire, that if traction were applied to an end of the device, and/or a small tear in the graft material was created, the entire device could unwind or “unzip” as a single nitinol-ePTFE/FEP thread. For these reasons it is noted there are a number of potential means by which the device could be safely retrieved, whether by pushing or pulling the stent graft out, or by partially or completely “unwinding” or “unzipping” the stent graft. Further, these methods are relatively unconstrained by approach (i.e. percutaneous, endoscopic, or surgical) allowing flexibility and redundancy in retrieval approaches. Again, such principles apply to devices of similar construction and are not limited to retrieval in the biliary system but instead can in theory be removed in any setting or situation.

It has been found that the direction and flexibility of the anchoring fins did have an impact on ease of device retrieval. Although there was only one minor complication directly related to device retrieval, devices in which the majority of the anchoring fins were oriented in the same direction as the vector of force for extraction, generally had easier extractions. For a percutaneous retrieval, this would entail devices that were deployed with the 3 layers of anchoring fins directed towards the liver (percutaneous delivery of 40 or 75 cm delivery shaft devices or endoscopic delivery of a 195 cm delivery shaft device). Further, noted that devices that were completely covered were also easier to remove by this mechanism than fenestrated stent grafts.

While the use of this device described heretofor was investigated primarily in the management of pre-existing strictures of varying etiology, it could just as readily be applied to a broad number of different situations. For example, given the natural history of the bile duct's healing response to injury, one potential application could be for the prevention of benign biliary strictures, such as with pancreatitis or iatrogenic injury. In this setting a removable stent graft could serve not only as a biliary conduit but also as a large diameter temporary scaffold around which the duct could remodel or “heal.” Similarly, a retrievable stent graft could also be applied to lesions that are incompletely characterized diagnostically at the time of intervention, but where there is still a need for biliary decompression. Finally, in the subset of patients that have malignant disease but a long life expectancy, the device could be periodically “exchanged” to combat intra-ductal sludge accumulation or stent edge overgrowth from mucosal hypertrophy or slowly progressive tumor extension. In general, these types of applications would all likely require longer implantation times, in the order of 3 to 6 months. Accordingly, given the potential role for stent graft retrieval in the management of biliary disease more, study is indicated to evaluate longer times to retrieval than currently evaluated as well as into the natural history of ductal disease post retrieval. Clearly, as has been stressed, the invention described here can be applied to any situation in which a stent or a stent graft can be used.

All or some of the following modifications can be made to make stent grafts easier to be removed:
1. removal or reduction in number of anchoring fins from stent graft;
2. primary modification of device and/or anchoring fins such that the fins are more flexible, easily invertible, collapsible, or compressible, in any of the 3 axis' or combination of the 3 axis' so as to minimize trauma against the duct or lumen wall and improve the removal profile of the stent graft when removing, moving or repositioning the stent graft;
3. Decreasing or broadening the angle of any or all of the anchoring fins at their apices or at their bases or both;
4. modifying one or both ends of the stent graft such that the stent component, graft component, or the entire stent graft (both stent and graft components) can more easily collapse or prolapse to assume a smaller and more compliant shape during the removal process than when deployed.
5. perforations or serrations embedded in the stent graft, or equivalent, that allow for more easy unzipping or unwinding of the stent graft;
6. beads, free edges, or divots at one or both ends of the stent graft that are either part of the stent, the graft component, or both that allows for or facilitates primary initiation of unzipping of the stent graft either by grabbing and pulling on them allowing for easier unzipping or unwinding of the stent graft or by placing a mandril-like device through the lumen of the stent graft which then catches the above beads, free edges, or divots and allows for the easier unwinding or unzipping of the stent graft through the primary action of the mandril on the stent graft; and
7. coating of the stent graft with pharmacologic or radioactive compounds for targeted and controlled release of said compounds whether therapeutic, diagnostic or both to the surrounding environment to prevent tissue growth in our around the stent graft

In addition to modifying the known stent grafts as noted above, better designed forceps will add in the removal of the stents. Such modifications to the forceps can include one of more of the following;
1. steerable with respect to the forceps component and/or the entire forceps device;
2. flexible with respect to the shaft;
3. the shaft can become rigid when needed that is controlled at the hand lever;
4. forceps that are atraumatic to the native lumen wall; and
5. forceps that can grasp, from within the lumen (intraluminal deployment), either partially or circumferentially, the stent graft.

Basic Indications for Stent Removal

Whereas the stent is currently indicated solely for treatment of malignant biliary strictures in the case of the Viabil Biliary Endoprosthesis, or in tracheobronchial strictures in the case of the Fluency for example, with the ability to retrieve stent grafts, now all patients with all manner of diseases in which a stent graft can be place, can now be potentially treated with these types of devices, whether treating malignant or benign strictures or providing a scaffold or serving as a temporizing measure in patients when no stricture is present in the hopes of preventing a stricture from forming, or providing a scaffold for remodeling, or as a means of locating the bile duct or other conduit during other procedures such as surgery or endoscopy. The stent can be removed by pulling on the device through the duct or lumen and into the funnel-end of an introducer sheath. While pulling, the anchor fins, if present, are allowed to flex backward and invert.

Removal of the stent generally involves gaining access to the device suitable for a subsequent traction force to be applied for removal, whether by unzipping, unwrapping or snaring or gaining access around the circumference of the endoprosthesis (whether from the outside of or from within the lumen of the stent graft) or by grabbing an edge of the endoprosthesis wall via a forceps-like or similar grasping device, again whether from an intra- or extraluminal approach or some combination of these methods. A general consideration when anticipating removal of the VIABIL Biliary Endoprosthesis® or any other stent is the capturing mechanism (whether snare, forceps etc) for access to an end of the device in vivo. For cases where the removal device is to be applied outside the stent graft there must be enough space to get one side of the removal device between the wall of the device and the wall of the duct. For cases where the removal device is to have a significant component that is within the stent graft, such considerations are not as critical.

Basic Approach and Considerations for Endoscopic Removal
In general, in cases where devices are deployed in the biliary system, devices which protrude through the ampulla are easier to access and capture via snare or forceps than those stent grafts that are isolated in the biliary system. Devices which are high up in the bile duct present a considerable challenge to access and capture by endoscopic approaches. In these cases, usually grasping forceps is the tool of choice. Alternatively, if there is also percutaneous access, a rendezvous approach can be utilized in order to guide endoscopic retrograde cholangiopancreatography (ERCP) access into the biliary system. Device access and removal methods can consist of:
GI access via endoscopy with removal of the stent graft via snare or grasping forceps with percutaneous approach assistance. Percutaneous access with utilization of a balloon, dilator, or other similar device to push the stent graft distally towards the bowel can assist in this type of retrieval by an endoscopic approach. Percutaneous access to the bowel with wires or equivalent into the bowel to either guide or allow endoscopy to snare or grasp the stent graft as a means of guiding or directing endoscopic access through the ampulla of Vater and into the biliary system (“rendezvous” approach) can also be applied in this type of endoscopic approach.
Direct access and removal via endoscopy without percutaneous approach assistance. This could be performed soley by endoscopic approach through the use of a snare or forceps to access the stent graft protruding through the papilla utilizing endoscopic visualization with removal of the stent graft through the mouth or pushing it distally into the bowel. It could also be performed by direct retraction of the stent graft through and into the working channel of the endoscope.

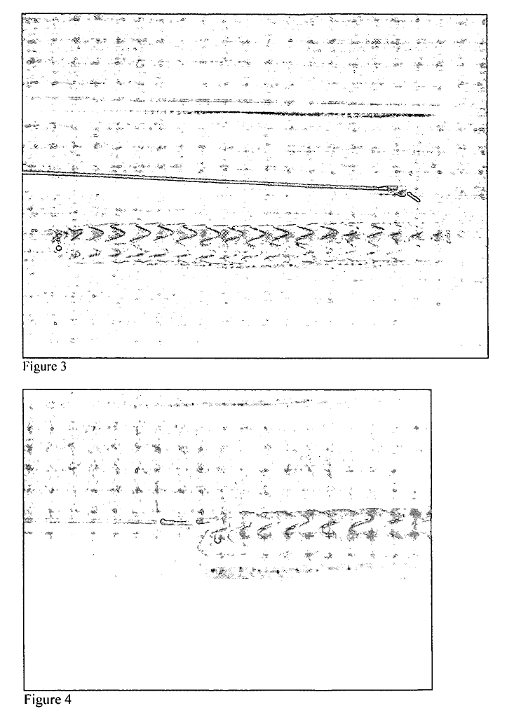

Basic Approach and Considerations for Percutaneous Removal (FIGS. 3 and 4)
Given utilization of the proper tools and techniques, percutaneous device removal may be a reasonably safe and easy removal alternative. The following describes one option of tools and technique possible. The basic concept is to reshape the end of the introducer sheath with a PTA balloon so as to fashion a funnel which aids in retraction of the endoprosthesis into the tube of the sheath. This is not mandatory but can aid in the more facile passage of the stent graft into the sheath. Utilizing an ERCP style, flexible shaft, Shark tooth forceps provides reliable capture of the edge of the stent graft. The Shark tooth forceps grasper jaw design is unique in that it allows perforation of the stent graft liner and then provides an overlapping, “C” shaped tooth to capture the stent wire. This capturing/jaw design then transfers the traction force to the internal side of the forceps tooth and minimizes the clamping force necessary to maintain capture of the stent graft by the forceps. These forceps have been more productive at stent graft removal, but are not the only forceps that can accomplish retrieval. Similarly, any device that can be used to grasp the stent or a portion of the stent can be used.

Basic Tools Used
1. Sheath
2. Percutaneous Transluminal Angioplasty (PTA) balloon or equivalent inflatable balloon type device
3. Forceps or snare or similar grasping device

Physician Description on Percutaneous Removal Technique:
1. Access biliary tree and insert guide wire as appropriate.
2. Perform imaging and cholangiogram to confirm appropriate position and retrievability of stent graft device
3. Insert appropriately sized diameter and length introducer sheath w/obturator.
   a. place sheath co-axially within the stent graft
   b. or, place sheath proximal to the free stent graft edge
4. Create a funnel at the distal end of the introducer sheath to aid pulling the stent graft into the introducer sheath tube; insert an appropriately sized high pressure, compliant, semi or noncompliant PTA balloon with a portion of the balloon in the sheath and the remainder of the balloon past the distal end of the sheath and inside the proximal end of the stent graft.
5. Utilizing an insuflater, inflate the balloon until full profile has been achieved. This usually requires approximately 12-15 atm. During balloon inflation, the balloon will want to “watermelon seed” and advance forward out of the sheath. Firmly pinching the balloon catheter shaft at the origin of the introducer sheath hemostatic valve will prevent the balloon from inadvertently advancing out of the sheath. Remove balloon. This will form a trumpet at the end of the introducer sheath which acts like a ramp for the stent graft to be pulled into the introducer sheath.
6. At this point the guidewire may be removed or left in place. Insert the grasping forceps into the introducer sheath and advance.
   a. if the sheath is inside the stent graft, retract the sheath until it is outside of the stent graft and of sufficient distance to allow the forceps to appropriately open and close unimpeded. Then open forceps and retract till just beyond the stent graft where it can now grasp the proximal edge.
   b. if the sheath is already proximal to the stent graft margin, advance forceps beyond sheath and up to the proximal stent graft edge and open.
7. Engage grasper jaws of the forceps over the wall of the stent graft (preferably grasping only one wall of the stent graft such that one tooth is on the outside of the stent graft and one tooth on the inside of the stent graft). If the stent graft is a tight fit in the duct and the edge of the stent graft is difficult to capture, an over-sized balloon can be used to over dilate the stent graft and surrounding duct. After balloon deflation, the stent graft diameter will retract, providing space between the duct and stent graft outer wall enabling the graspers outer tooth to slip between the stent graft adduct wall. While maintaining grasper jaw engagement of stent graft wall, withdraw forceps until stent graft pulls into introducer sheath. Once the stent graft is retracted into the introducer sheath, either pull the stent graft into and subsequently through the sheath or pull the sheath, with the stent graft inside, both out of the patient (this preferably is done co-axially over a wire so as to maintain access).
8. Alternatively, a flexible sheath can be used which can be applied as described above in steps 3 and 6 (the sheath tip can also be “trumpeted” as described in steps 4 and 5 if necessary) except that once the stent graft is grasped with the forceps as described in step 7, the sheath is instead gradually advanced forward in a co-axial manner over the stent graft while applying traction on the forceps so as to gradually engulf the captured stent graft with the forward projection of the sheath.
9. Similarly, measures can be applied for unzipping the stent graft. When the margin is grasped with the forceps and sufficient force or traction is applied to the forceps, the stent graft can also unwind or unzip. When applying an unzipping or unwrapping approach, sufficient traction must be applied to initiate the unzipping process. In such cases, when applying this approach to the VIABIL Biliary Endoprosthesis, the device will then unwrap or unzip as an FEP/ePTFE covered nitinol wire (FIG. 5). This method of removal (unzipping or unwinding) can be aided by the application of a counterforce through the use of an inflated balloon angioplasty catheter placed and inflated in the stent graft lumen which holds the graft in place or by applying forward pressure on the inflated balloon, while apply a pulling force on the forceps. In addition, if perforations or a perforated edge or margin in the stent graft is created with the purpose of possible future unzipping the stent graft to primary placement of the stent graft in the patient, then the unzipping process is significantly aided and less of a counterforce is needed. Further, if a perforated edge is already present in either edge of the stent graft (the proximal or distal edge of the stent graft), and this edge can be grasped, such as with the aid of a snare device or grasping forceps, then the unzipping process is further facilitated as this edge, margin, or bead can serve as the initiation point for the unzipping process.
10. The method described above in number 9 for unzipping the stent graft can also be applied in the same manner when using an endoscopic approach.
11. Intraluminal retrieval or snaring: use of a device that is capable of grasping the stent graft from either with in the lumen of the stent graft outside of the stent graft, or in some combination and is able to grasp the stent graft in either a discrete or circumferential manner. This is one method that will also facilitate collapsing or compressing the stent graft into the retrieval sheath.
12. Balloon—snare retrieval technique: this method describes using a device which allows a retrieval snare to lasso the stent graft. Briefly, this method employs placing a balloon or some equivalent device coaxially through the stent graft which serves as a “ramp” thereby allowing a snare to form and ensnare the stent graft. Once the stent graft is snared, it can be retrieved into the retrieval sheath using any number or combination of the methods described above.

These exact same principles for removal would apply to any situation in which a metal or plastic stent, drain or tube could be placed as described in the literature. The placement and retrieval method for this device would not significantly alter in principal from what has been heretofore described. With the ability to remove the stents, new uses for the stent in the biliary system can now include but are not limited to treatment or palliation of:
1.benign strictures
2.malignant strictures
3.strictures of unknown etiology
4.stricture prevention in patients who have or have suffered from:
   a. inflammatory bowel disease, or other inflammatory disease or conditions that primarily or secondarily effect the biliary system
   b. sclerosing cholangitis, or
   c. biliary cirrhosis, or
   d. pancreatitis, or
   e. prevention of duct strictures or injury when patients are undergoing therapy whether surgical, endoscopic, interventional, pharmacologic, or using radiation where the therapy can potentially primarily or secondarily injure the binary system
      5. external compression syndromes (e.g. lymph nodes, fluid collections, malignant or benign processes that externally compresses the biliary system)
      6. to serve as a biliary conduit—whether temporary or permanent
      7. duct localization in surgical or endoscopic or interventional procedures
      8. intraluminal complete or partial occlusions that are not due to strictures (e.g. foreign body, sludge, tissue overgrowth or ingrowth, stones, debris, or are of an iatrogenic nature)

In addition, the operator can now remove the stents for these instances.

Stents could be removed in the event of (1) unsatisfactory placement, whether primarily or secondarily, (2) device occlusion (food, foreign body, in-growth of tissue, overgrowth of tissue at STENT GRAFT margins, insispation with fluid, debris, cells, tissue, blood or bile or the like), (3) cholecystitis, (4) misdiagnosed (malignant to benign) stricture or lesion etiology, (5) desire to treat benign strictures, (6) infection, (7) a need to provide a temporary scaffold or conduit, (8), device malfunction or failure, (9) inappropriate device sizing, (10) a need for staging procedures, whether diagnostic or therapeutic. Stent grafts are not limited in use to the biliary system. This same method disclosed for stent graft retrieval can in principle be applied to any situation in which a stent graft could be placed as has been described in the literature. Such stent graft applications would include, but are not limited to:
1. Any portion of the alimentary canal—from the level of the esophagus through to the level of the rectum.
2. the tracheobronchial system
3. The genitourinary system—such as the ureters or urethra
4. the vascular system—whether arterial or venous

The following examples demonstrate the present invention.

### EXAMPLE 1

Patients:

Between December 2004 and October 2005 a 6 patients had Viabil Biliary Endoprostheses placed and retrieved (W. L. Gore Associates, Flagstaff, Ariz.). With respect to the patients described here, three patients had focal anastamotic strictures associated with previous orthotopic liver transplantation. One patient had a chronic recalcitrant focal stenosis of the distal left hepatic duct of uncertain etiology. One patient had a mucinous tumor of her biliary system. The final patient had unresectable metastatic adenocarcinoma involving both the right and left hepatic ducts extending into the proximal common hepatic duct. All patients initially presented with clinical findings consistent with biliary obstruction and infection and were subsequently decompressed with percutaneous biliary drainage and treated with antibiotics.

At the time of stent graft placement, all patients had internal/external biliary drains in place. Of the 3 patients with transplant-related strictures (patients 1, 3, 4), two patients had only left sided percutaneous access, while the other patient had only right sided access. Patient 2, who had the left hepatic duct stricture, had bilateral percutaneous biliary access, while patient 5 also had bilateral percutaneous access. The patient with the mucinous tumor (patient 6) also had right sided percutaneous access.

Device

The biliary stent graft used in these procedures consists of a nitinol exoskeleton with an expanded polytetraflouroethylene (ePTFE) and fluorinated ethylene propylene (FEP) tubular lining (FIG. 1). The device has 2 tiers of anchoring fins which are opposed in direction (one tier facing towards the bowel, the other tier directed towards the liver) and positioned at opposite ends of the stent graft. One tier has 3 layers of anchoring fins with 3 fins per layer (total of 9 fins) while the other tier consists of one layer of 3 fins at the other end of the device. In general, the device is meant to be deployed such that the tier with 3 layers of anchoring fins is directed towards the liver so as to maximize “anchoring” of the stent graft within the bile duct. However, these directionally opposed tiers of fins can be positioned such that the 3 layers of fins are pointing towards the liver (40 cm and 75 cm percutaneous delivery shaft device) or pointing towards the bowel (195 cm ERCP length delivery shaft) when employing a percutaneous delivery approach depending on the delivery shaft used. In addition, the biliary stent graft used in this study also has two forms of graft covering: one in which the stent graft is completely covered, and the other in which the proximal aspect has fenestrations in the graft material (FIG. 2).

Device Placement

Each patient received one stent graft, except patients 4, 5 and 6 who received 2 devices during their initial stent graft placement procedure. All devices were deployed through standard 10 French sheaths. Of the biliary endoprostheses described here, 2 were 10 mm×6 cm (195 cm delivery shaft length), 1 was 10×10 (40 cm shaft length), 1 was 10×6 (40 cm shaft length) and 5 were 8 mm×6 cm (1 195 cm shaft length, 3 40 cm shaft length, 1 75 cm shaft length). Patients 4 and 5 each received stent grafts with proximal fenestrations. Fenestrated stent grafts where used in patient 4 as these were the only appropriately sized Viabil stent grafts at available at the time of the procedure. In patient 5 however, fenestrated devices where used because of the central drainage of one of the biliary ducts into the right hepatic duct. Therefore, to avoid “jailing” this duct, two fenestrated stent grafts were placed across the right and left hepatic ducts allowing unimpeded drainage of the anomalous biliary duct. The remaining stent grafts employed (patients 1-3, 6) were covered along their entirety.

All devices were deployed after initial cholangiograms were performed in order to define ductal anatomy, lesion characteristics and to appropriately size the device. All patients underwent pre-dilation cholangioplasty as well as post deployment cholangioplasty except patients 5 and 6, in which both stent grafts were primarily deployed and no post deployment cholangioplasty was performed. Standard post stent graft deployment cholangiography was performed in all cases. Patients were given intravenous antibiotics immediately before the procedure. At the end of the procedure, all patients were left with internal/extemal biliary drains to gravity drainage.

Patients were monitored for 23 hour observation post deployment and then discharged home. All patients had their drains capped prior to discharge and were placed on oral antibiotics for gram negative coverage for 1 week. They were then seen in follow-up one week post-procedure to assess their biliary drainage status. The patients were then brought back for re-evaluation and follow-up cholangiogram and stent graft retrieval.

Device Retrieval

For device retrieval, all patients were placed on intravenous antibiotics pre-procedurally and oral antibiotics for 1 week post retrieval. A long 10 or 12 F sheath was placed through which the initial cholangiogram and stent graft retrieval was performed. A safety wire was concurrently placed into the small bowel for maintaining access. The primary method of stent graft retrieval in these descriptions was by placing in parallel to the safety wire, endoscopic grasping forceps (Olympus Medical, Orangeburg, N.Y.) which were used to grab the proximal edge of the stent graft. The stent graft was then pulled through the sheath by applying constant traction on the grasping forceps. Entry of the stent graft into the sheath was facilitated in some cases by flaring of the sheath tip with a 10 or 12 mm high pressure balloon appropriate to the sheath size. Slight progressive forward advancement of the sheath in order to “engulf” the stent graft once the proximal margin had been pulled into the sheath also facilitated retrieval in latter cases. In these cases, “flaring” of the sheath tip was not performed. Additionally, snaring of the proximal edge of the stent graft using a loop snare was also employed. In this case, the loop snare was advanced coaxially over an inflated balloon that was deployed partially within the proximal margin of the stent graft. The snare was thus able to form around and slide over the balloon and snare the outside of the stent graft. The snared stent graft was then retrived into the retrieival sheath using a combination of the techniques described above. A completion cholangiogram after stent graft retrieval was then performed and an internal/external biliary drain placed. Patients were then kept overnight for observation and maintained on antibiotics and sent home the following day with drainage to gravity.

Of the 9 total stent grafts placed, 8 underwent attempted retrieval. The first three devices were initially placed as a last effort to treat recalcitrant biliary strictures (patients 1-3). Because of the unknown natural history of biliary stent graft retrieval we decided to err on the conservative side of time to initial stent graft retrieval in these patients. One device was removed for initial misplacement (patient 4). Patient 5, in whom as previously described, fenestrated stent grafts were used so as not to cover the centrally draining bile duct demonstrated interval tumor growth through the stent graft fenestrations at 7 week follow-up. In addition, cholangiography now demonstrated occlusion of this centrally draining duct from tumor progression. Therefore, since there was no longer a need for fenestrations it was decided to remove the stent grafts at this time and replace them with completely covered stent grafts in the hopes of providing a better barrier to tumor progression. Finally, Patient 6 had their device removed due to significant device migration and stent graft to bile duct lumen size mismatch.

In total, all 8 stent grafts were successfully removed over a total of 7 separate procedures. In all cases, biliary stent grafts were extracted in their entirety. Post-retrieval cholangiograms for all 8 devices across all 5 patients were unremarkable as were their post-retrieval hospital courses.

All retrieval procedures were initially attempted in a retrograde (percutaneous) fashion over six extraction procedures for all six patients. This approach was technically successful with 7 out of 8 devices (patients 2-5). The remaining device (patient 1) was ultimately removed on a subsequent follow-up procedure via a combined anterograde (endoscopic) and retrograde approach in combination with endoscopic retrograde cholangiopancreatography (ERCP). During this extraction procedure, a rendezvous approach was ultimately required as the patient's endoscopic ampullary cannulation was difficult secondary to altered anatomy and previous sphincterotomy. Once endoscopic access was obtained from below, the stent graft was then snared using ERCP grasping forceps deployed via the ERCP scope. The stent graft was then pulled in toto from the duct and into the bowel and then out of the patient in tandem with the scope.

The mean stent graft retrieval procedure time was approximately 40 minutes across all 8 stent graft extractions with a range of 10 minutes (patient 4) to 210 minutes (patient 1: combined time across two separate extraction procedures). This was defined as the time from which actual stent graft extraction was initiated to actual ex vivo removal of the entire device from the patient. The mean extraction time for percutaneous extraction attempts was around 33 minutes.

As the primary focus of this study was to evaluate biliary stent graft retrieval: two categories of procedural-related complications: those directly related to stent graft retrieval and those unrelated to stent graft retrieval aer reported. Under the direct category, report one minor complication resulting from stent graft retrieval in the immediate peri-procedural period which consisted of a small amount of tract bleeding near the skin entry site that was treated with a small amount of gel foam and placement of a 14F biliary drain. In the unrelated category, it is reported 3 complications. During the primary stent graft placement procedure, patient 2 developed a biliary-hepatic arterial fistula in the contralateral side to which the stent graft was deployed which was successfully treated with coil embolization. Patient 3 experienced an episode of presumed septicemia immediate post-stent graft placement from which he recovered uneventfully. Finally, patient 4, as previously described, had incorrect placement of the initial device. In this case, the initial stent graft was overextended into the left hepatic duct and covered a prominent intrahepatic duct prompting immediate removal and placement of a second device. The remaining patients had uncomplicated stent graft deployment and retrieval procedures.

In conclusion, here is reported initial experience with retrieval of the Viabil stent graft from the biliary system. Retrieval was technically successful in all patients although the first patient required a staged procedure. Success was not affected by underlying disease etiology which included strictures associated with benign disease, malignant disease, disease of unknown etiology, device migration, as well as a device misplacement. Success was not affected by tine of stent graft implantation. Finally, stent graft retrieval demonstrated a steep learning curve and further, could be accomplished with both anterograde and retrograde approaches.

The Fluency tracheobroncial stent graft® by Bard, is a covered stents wherein flexibility of the stent is retained. Further detailed in U.S. 20030191519 and 20010039446 and 20040236400. Given that the Fluency stent graft is similar in concept to the Viabil stent graft in that it is also composed of a graft component and a shape memory stent component, the exact same principles for retrieval would apply as disclosed in this invention except the ability to unzip the stent graft as the Fluency stent graft stent and graft components are not constructed in the same fashion as the Viabil stent component. Thus, unzipping of the Fluency Stent graft would not be as feasible as with the Viabil in this manner of retrieval.

### EXAMPLE II

Removal of Stent Grafts From the Vasculature

Two patients with end-stage renal disease with an arterial-venous graft had covered stents placed because of a recurrent, recalcitrant stenoses that demonstrated repeated restenosis and high elastic recoil despite repeated balloon angioplasty near the venous outflow. In both patients, the stent grafts used was a Viabahn stent graft from W L Gore which was placed in order to serve as a vascular conduit across the recalcitrant stenoses and to also serve as a scaffold for the vessel to remodel around. The first patient subsequently returned for extraction of the implanted stent graft. Using a similar technique as described and detailed above the stent graft was removed approximately 3 weeks later after initial stent graft placement. Namely, in this patient bidirectional vascular access was obtained surrounding the stent graft (a 10 French sheath was placed distal to the proximal margin of the stent graft directed towards the inflow of the graft, and a second 10 French sheath was placed distal to the distal margin of the stent graft directed toward the outflow of the graft). Using grasping forceps through both sheaths, both margins (proximal and distal ends) of the stent graft were captured. Using a combination of “unzipping” (by applying opposed traction on both ends of the stent graft through the two grasping forceps to unzip the stent graft), and pulling of the stent graft into the distally placed sheath, while simultaneously advancing this sheath to “engulf” the captured stent graft, the entire stent graft was ultimately removed (FIG. 5a-c). As with the previous descriptions above, the vascular stent graft could be removed by the same methods as previously detailed. In the second patient, the stent graft was removed in similar fashion. Namely, the patient returned approximately 4 weeks after initial deployment for removal. Access to the stent graft was obtained in a single direction in this instance, namely proximal to the inflow to the stent graft. A 10 French sheath was placed through which grasping forceps were used to grab the proximal end of the stent graft. Using similar technique as previously described, the stent graft was removed in toto through a combination of pulling and partially unzipping the stent graft while simultaneously engulfing it through progressive advancement of the sheath over the stent graft. In both cases, the stent grafts were removed in toto and there were no complications from stent graft removal. Similarly, this method would apply to any stent graft and is not inclusive to the Viabahn, Viabil or Viator vascular stent grafts from W L Gore.

Applications of this method could apply to any scenario where a stent graft could be used in the vascular system whether arterial or venous or arterial-venous conduits.

All references including patents and applications and ® products cited above are expressly incorporated by reference in their entirety as if fully written herein.

### Biliary Disease Management and Next Steps: Biliary Stent Graft Retrievability

Those skilled in the art will understand and appreciate that while the present invention has been described with reference to its preferred embodiments and the examples contained herein, certain variations in material composition, shape memory alloy constitution, stent and ePTFE dimensional size and configuration, temperatures, times and other operational and environmental conditions may be made without departing from the scope of the present invention which is limited only by the claims appended hereto. For example, one skilled in the art will understand and appreciate from the foregoing that the methods for making each of the foregoing embodiments differs with each preferred embodiment. These differences in the methods are largely due to the selection of intraluminal stent type and whether the intraluminal stent is intended to be removed in a matter of days, weeks or months.

I claim:

1. A removable intraluminal stent graft comprising:
a) an intraluminal stent graft which is capable of unzipping when traction is applied in one direction and little sufficient force is applied in the other direction.

2. A method of removing an intraluminal stent graft from a patient comprising:
a) Grasping said intraluminal stent graft;
b) Unzipping said intraluminal stent graft; and
c) Pulling or pushing said unzipped intraluminal stent graft from said patient.

3. The method as recited in claim 2 wherein said intraluminal stent graft is self expanding.

4. A method of removing a self expanding non stainless steel intraluminal stent graft from a patient comprising:
Grasping or snaring said self expanding non stainless steel intraluminal stent graft; and
Pulling or pushing said self expanding intraluminal stent graft from said patient percutaneously, surgically, or endoscopically.

5. The method as recited in claim 4 further comprising advancing or retracting said self expanding non stainless steel intraluminal stent graft into a sheath.

6. The method as recited in claim 5 wherein said stent graft is pulled or pushed through or into said sheath.

7. The method as recited in claim 5 wherein said sheath and said stent graft are pulled or pushed out of said patient separately or together.

8. The method as recited in claim 4 wherein said non stainless steel is a memory alloy.

9. The method as recited in claim 8 wherein said memory alloy is nitinol.

10. The method as recited in claim 4 wherein said stent graft is substantially non-migrating.

11. A method of removing a self expanding intraluminal stent graft from a patient comprising;
a) advancing a mandril through or within said self expanding intraluminal stent graft;
b) affixing said self expanding intraluminal stent graft and said mandril together;
c) compressing or rewrapping said self expanding intraluminal stent graft around said mandril; and
d) pulling or pushing said mandril and self expanding intraluminal stent graft from said patient.

12. A method of treating strictures, stenoses, obstructions or complete or partial occlusions in a patient in need thereof, comprising;
inserting a stent graft into a patient; and
removing said stent graft from said patient.

13. The method as recited in claim 12 wherein said stent graft is a biliary endoprosthesis.

14. The method as recited in claim 13 wherein said biliary endoprosthesis is a VIABIL® biliary endoprosthesis.

15. The method as recited in claim 12 wherein said strictures, stenoses, obstructions or complete or partial occlusions are in a blood vessel or blood conduit.

16. A method of treating strictures, stenoses, recurrent stenoses, obstructions, or complete or partial occlusions in a patient in need thereof, comprising;
inserting a VIABIL® biliary endoprosthesis into blood vessel or blood conduit; and
removing said VIABIL® biliary endoprosthesis from said patient.

17. The method as recited in claim 16 wherein said stenoses, obstructions or occlusions consist of or are due to:
a. atherosclerotic or atherosclerotic related-lesions
b. calcific lesions
c. restenosis
d. a fibrotic reaction
e. an inflammatory response
f. blood clot of either an acute, subacute or chronic nature
g. compression from a force that is extrinsic to the blood vessel whether benign or malignant
h. intimal hyperplasia
i. neointimal hyperplasia
j. an embolus
k. a result of fibrodysplasia
l. neoplasia either benign or malignant of the blood vessel or its components
m. Infection
n. trauma
o. vasospasm
p. drug-induced
q. dissection
r. congenital
s. medial hyperplasia
t. adventitial hyperplasia
u. iatrogenic injury
v, radiation

18. A method of treating a disorder in a patient, comprising;
inserting a stent graft into a blood vessel or blood conduit; and
removing said stent graft from said patient wherein said disorder is selected from the group consisting of aneurysms, pseudoaneurysms, vascular dissections, vascular malformations, injuries to blood vessels that do or could result in bleeding or extravasation of blood, porto-systemic shunts and vascular dialysis conduits.

19. The method as recited in claim 18 wherein said vascular malformation is either primary or secondary in etiology and predominantly effects either the arteries, veins, or both.

20. The method as recited in claim 18 wherein said porto-systemic shunt is an intrahepatic shunt.

21. The method as recited in claim 18 wherein said stent graft is the VIATOR®, Viabil, or Viabahn stent graft.

22. The method as recited in claim 18 wherein said dialysis conduit is an arterial-venous fistula.

23. The method as recited in claim 18 wherein said dialysis conduit is an arterial-venous graft.

24. A method of creating an intentional occlusion in a blood vessel or blood conduit in a patient in need thereof, comprising;
inserting a stent graft into a blood vessel or blood conduit; and
removing said stent graft from said patient.

25. The method as recited in claim 13 comprising;
inserting said VIABIL® biliary endoprosthesis into a bile duct; and
removing said VIABIL® biliary endoprosthesis from said patient.

26. The method as recited in claim 12 wherein said strictures is benign.

27. The method as recited in claim 12 wherein said strictures is malignant.

28. The method as recited in claim 31 wherein said strictures is of unknown etiology.

29. A method of treating non-liver transplant benign strictures in a patient in need thereof, comprising;
inserting a stent graft into a patients bile duct; and
removing said stent graft from said patient.

30. The method as recited in claim 29 wherein said stent graft is a VIABIL®.

31. A method of treating benign strictures in a liver transplant patient in which said benign strictures is caused by non-iatrogenic factors, comprising;
inserting a stent graft into a patients bile duct; and
removing said stent graft from said patient.

32. The method as recited in claim 31 wherein said stent graft is a VIABIL®.

33. A method of preventing or treating biliary strictures in a patient in need thereof comprising; inserting a stent graft into a patients bile duct; and
removing said stent graft from said patient when the body's healing process has matured or when the inciting agent predisposing to stricture formation is mitigated.

34. The method as recited in claim 33 wherein said stent graft is a VIABIL®.

35. The method as recited in claim 33 wherein said strictures is caused by external compression syndromes.

36. A method achieving an effect in a patient comprising;
inserting a stent graft into a patients bile duct;
removing said stent graft from said patient: and
wherein the effect is providing duct localization, treating bilary leaks or fistulas or treating complete or partial biliary occlusions,

37. The method as recited in claim 36 wherein said complete or partial biliary occlusions are selected from the group consisting of:
1. foreign body;
2. debris;
3. sludge;
4. stones;
5. tissue accumulation or deposition as the result of the bodies response to a perturbing force;
6. bodily tissue in-growth or at the margins or edges of an already placed stent, stent graft, surgical graft, or surgical conduit whether native or artificial; and
7. resulting from or of an iatrogenic nature.

38. The method as recited in claims 36 wherein said stent graft is a VIABIL®.

39. The method as recited in claim 36 wherein said stent graft is a biliary stent graft.

40. A method of treating strictures of the respiratory, gastrointestinal or genitourinary system, comprising; inserting a stent graft into a patient's respiratory, gastrointestinal or genitourinary system; and removing said stent graft from said patient.

41. The method as recited in claim 40 where the respiratory system is the tracheobronchial system

42. The method as recited in claim 40 where the gastrointestinal system is the oropharynx, esophagus, stomach, duodenum, small intestine, large intestine and rectum or any surgical reconstruction or revision thereof.

43. The method as recited in claim 40 where said stricture is benign.

44. The method as recited in claim 40 where said stricture is malignant.

45. The method as recited in claim 40 where said stricture is of unknown etiology.

46. The method as recited in claim 40 where the genitourinary system is the kidneys, ureters, bladder, or urethra or any surgical reconstruction or revision thereof.
